(19) United States
(12) Patent Application Publication
(10) Pub. No.: US 2009/0286865 A1
(43) Pub. Date: Nov. 19, 2009

(54) POTENTIATOR OF ANTITUMORAL AGENTS IN THE TREATMENT OF CANCER

(75) Inventors: Andre PICHETTE, Chicoutimi (CA); Jean Legault, Chicoutimi (CA)

Correspondence Address: BIRCH STEWART KOLASCH & BIRCH, PO BOX 747, FALLS CHURCH, VA, 22040-0747, US

(73) Assignee: F.P.L. PHARMA INC., Montreal (CA)

(21) Appl. No.: 12/510,196

(22) Filed: Jul. 27, 2009

Related U.S. Application Data
(63) Continuation of application No. 10/488,682, filed on Apr. 6, 2004.

(30) Foreign Application Priority Data
Sep. 5, 2001 (CA) 2356438

(51) Int. Cl. A61K 31/337; A61K 31/01; A61P 35/00

(52) U.S. Cl. 514/449; 514/762

(57) ABSTRACT

The present invention relates to a potentiator composition for enhancing therapeutical effect of an antitumoral agent, said composition comprising a terpene or derivative thereof in association with a pharmaceutically acceptable carrier.

### BACKGROUND OF THE INVENTION

1. Field of the Invention

This invention relates to the use of terpenes and derivatives thereof as a potentiator of antitumoral agents in the treatment of cancers.

2. Description of Prior Art

The toxicity induced by anticancer agents is a major problem during cancer treatments. The use of no toxic potentiator or synergistic compounds in combination with anticancer agents may greatly potentiate their effectiveness while avoiding the toxicity. Until now, the most of protocol of clinical cancer treatments use a combination of several anticancer agents which act in synergism, for example, MVAC protocol constituted of four drugs including methotrexate, vinblastine, doxorubicin and cisplatin. However, usually each compound of these cocktails is toxic as well as their combination.

The terpenes are a class of natural compounds widely distributed in nature, mostly in the plant kingdom. This class of compounds could play an important role in the chemical defense against pathogens and herbivores (Cates, R. G. Recent Advances in Phytochemistry. 1996, 30: 179-188). Some terpenes have also many biological and pharmaceutical activities, which can be useful to treat human diseases. For example, the volatile terpenes as monoterpenes and sesquiterpenes are known to have several pharmacological activities including antibacterial, antifungal, antispasmodic, sedative and analgesic (G. Buchbauer, L. Jirovetz. Flavours and Fragrances Journal 1994, 9, 217-222; R. Teranishi, et al. American Chemical Society. 1993, vol 525; J. Bruneton. Pharmacognosie, 3e edition. Editions Tec & Doc, Paris, 1999). Moreover, some diterpenes shown to have an antitumoral, antihypertension, anti-inflammatory and analgesic activities (J. Bruneton. Pharmacognosie, 3e edition. Editions Tec & Doc, Paris, 1999; J. R. Hanson. Natural Product Reports. 1998, 15, 93-106). The triterpenes are known to have the following activities: antiviral, antibacterial, antitumoral, anti-inflammatory, molluscicide, analgesic, hypocholesterolemic and insecticide (J. Bruneton. Pharmacognosie, 3e edition. Editions Tec & Doc, Paris, 1999; S. B. Mahato, et al., Phytochemistry. 1992, 31, 2199-2249; S. B. Mahato, S. Sen. Phytochemistry. 1997, 44, 1185-1236). The literature report that the monoterpenes such as limonene and perillyl alcohol, may act synergistically with antiestrogens and retinoids (Lackey, B. R., et al. Medical Hypotheses. 2000, 54: 832-6) but these ones are active when used alone and are also cytotoxic. Moreover, some triterpenes isolated from Panax and Glycyrrhiza (Hasegawa H, et al. Planta Med 1995, 61: 409-413) and a sesquiterpene isolated from Torilis japonica (Kim S E, et al., Planta Med 1998, 64: 332-334) are known to reverse multidrug-resistance in cancer cells and to enhance the cytotoxicity of several anticancer agents. However, these terpenes do not enhance the cytotoxicity of anticancer agent in sensitive cell lines. Moreover, Benet et al., showed that various essential oil can increased bioavailability of an orally administered hydrophobic pharmaceutical compound by inhibition of cytochrome P450 and/or decreasing of P-glycoprotein drug transport (U.S. Pat. No. 5,916,566).

It would be highly desirable to be provided with terpenes and terpenes derivatives to be used as potentiators of antitumor agents which are non-toxic.

### SUMMARY OF THE INVENTION

The present invention relates to the use of terpenes and derivatives thereof as a potentiator of antitumor agents. The invention could be used also for the immunotherapy and genetic therapy and all other cancer treatments.

Terpenes can be co-administrated with antitumor agents in humans or in other animals as pharmaceutical composition, a foodstuff or a dietary supplement, to treat or prevent cancer.

In accordance with a preferred embodiment of the present invention there is provided a potentiator composition for enhancing therapeutical effect of an antitumoral agent, the composition comprising a terpene or derivative thereof in association with a pharmaceutically acceptable carrier.

In accordance with a preferred embodiment of the present invention the terpene is selected from the group consisting of monoterpene, diterpene, sequiterpene and triterpene.

In accordance with a preferred embodiment of the present invention the terpene is β-caryophyllene.

In accordance with a preferred embodiment of the present invention the composition further comprises an antitumoral agent.

In accordance with a preferred embodiment of the present invention the composition of the antitumoral agent is from a class selected from the group consisting of alkylating agent, antimetabolite, antimitotic, antibiotic, immunotherapy and hormone.

In accordance with a preferred embodiment of the present invention the composition of the alkylating agent is selected from the group consisting of melphalan, cyclophosphamide, lomustine, carmustine and cisplatine.

In accordance with a preferred embodiment of the present invention the composition of the antimetabolite is selected from the group consisting of 5-fluorouracil, cytarabine and methotrexate.

In accordance with a preferred embodiment of the present invention the composition of the antimitotic is selected from the group consisting of paclitaxel, vincristine, vinblastine and vindesine.

In accordance with a preferred embodiment of the present invention the composition of the antibiotic is selected from the group consisting of doxorubicin, aclarubicin, daunorubicin and mitomycin C.

In accordance with a preferred embodiment of the present invention the composition of the immunotherapy is selected from the group consisting of vaccine, cytokine and interleukine.

In accordance with a preferred embodiment of the present invention the composition of the hormone is selected from the group consisting of steroid and glucocordicoid hormone.

In accordance with a preferred embodiment of the present invention the composition of the preferred antitumoral agent is paclitaxel.

In accordance with a preferred embodiment of the present invention a method for enhancing the therapeutical effect of an antitumoral agent in a patient comprising the step of administering a therapeutically effective amount of the potentiator composition of the present invention in combination with the antitumoral agent to said patient.

In accordance with a preferred embodiment of the present invention the method of the administering of the composition is simultaneous, together or separately, consecutively and shortly prior to or after administering the antitumoral agent.

In accordance with a preferred embodiment of the present invention a method for enhancing the effect of an antitumoral agent in a patient comprising the step of administering a therapeutically effective amount of the composition of the present invention to the patient.

In accordance with a preferred embodiment of the present invention the method of the administering is performed intravenously, orally, intraperitoneally, topically, subcutaneously, transdermally, intramuscularly, nasally, aerosolly, rectally and sublingually.

For the purpose of the present invention the following terms are defined below.

The term “terpene” is intended to mean, without limitations, mono-, di-, sesqui-, triterpenes and all related derivatives as well as a mixture of these compounds.

The term “potentiator” is intended to mean a compound that increases the efficiency or enhance the therapeutical effect of at least one other compound (antitumoral agent) administered simultaneously, together or separately consecutively and shortly prior to or after whereby the combined action is greater than the sum of separate, individual actions.

The term “Antitumoral agent” is defined as any substance intended for use in the treatment or the prevention of cancer, or in the reduction of a tumor size, or in the reduction of a tumor growth, including also any antitumor agents.

### BRIEF DESCRIPTION OF THE DRAWINGS

FIG. 1 illustrates the synergistic effect of β-caryophyllene with paclitaxel in human breast adenocarcinoma MCF-7 cell lines;

FIG. 2 illustrates the synergistic effect of β-caryophellene with paclitaxel in DLD-1 cell line;

FIG. 3 illustrates the synergistic effect of β-caryophyllene with paclitaxel in L-929 murine cell line; and

FIG. 4 illustrates the synergistic effect of β-caryophyllene with paclitaxel in human fibroblasts.

### DETAILED DESCRIPTION OF THE INVENTION

In accordance with the present invention, there is provided terpenes and terpenes derivatives suitable to be use as potentiators of antitumoral agents for the treatment of cancer.

The screening of terpene potentiators in accordance with the present invention is carried out by the evaluation of several parameters including: i) the cytotoxicity (maximal tolerated dose); and iii) the effect on the intracellular glutathione content; and iv) the synergism in combination with antitumor agents. Briefly, the cytotoxicity or the cell growth inhibition induced by the terpenes is evaluated on various cell lines in order to determine the maximal tolerated dose (MTD) or non-toxic dose. The MTD is the higher concentration that does not induce cell growth inhibition, for example, the MTD for β-caryophyllene is greater than 800 μM. The cell growth is measured by the fluorescence induced by the metabolic transformation of Resazurin™ in Resorufin™, which is proportional to living cells (see materials and methods). As showed in Table 1, β-caryophyllene does not induce cytotoxicity against normal and cancerous cell lines tested in vitro. Moreover, β-caryophyllene is not toxic in mice at >500 mg/kg. It is shown herein that β-caryophyllene is having a synergistic action on the treatment of cancer when used in combination with antitumoral agents such as paclitaxel. To assess the synergistic effect of terpenes such as β-caryophyllene, the cancer cells are treated with growing concentrations of antitumoral agents (paclitaxel) with or without terpene (β-caryophyllene). The evaluation of the synergistic effect is described in materials and methods of Example I.

TABLE 1
Maximal tolerated dose (MTD) of β-caryophyllene
on normal and various tumor cell lines

| Tissue | Cell lines | MTD (μM)^{a} |
|---|---|---|
| human breast adenocarcinoma | MCF-7^{b,c} | >800 |
| human prostatic | PC-3^{b,c} | >800 |
| adenocarcinoma | | |
| human lung carcinoma | A-549^{b,c} | >800 |
| human colon adenocarcinoma | DLD-1^{b,c} | >800 |
| human melanoma | M4BEU^{d} | >800 |
| human fibroblast | fibroblast^{e} | >800 |
| mouse subcutaneous | L-929^{b,c} | >800 |
| connective tissue | | |

^{a}Maximal tolerated dose (MTD) or maximal no toxic dose.
^{b}ATCC: American Type Culture Collection.
^{c}ECACC (Salisbury, United-Kingdom): Europeen Collection of Cell Culture.
^{d}Biopredic International (Rennes, France).

The results presented herein show that β-caryophyllene increases cell growth inhibition, induced by the paclitaxel, of about 40% in a human breast adenocarcinoma MCF-7 cell lines.

The present invention will be more readily understood by referring to the following examples which are given to illustrate the invention rather than to limit its scope.

### Example I

### β-Caryophyllene as Synergist in Combination with Paclitaxel (Taxol®) Against MCF-7 Cell Lines, a Human Breast Adenocarcinoma

### Material and Methods

### Cell Culture

The human cell lines breast cancer adenocarcinoma MCF-7, prostatic adenocarcinoma PC-3, lung carcinoma A-549 and colon adenocarcinoma DLD-1 together with the mouse cell line L-929 (fibrobast) were obtained from the European Collection of Cell Cultures (ECACC, Salisbury, United Kingdom). Normal human fibroblasts were purchased from Biopredic International (Rennes, France). The M4BEU human melanoma cell line was generously supplied by Dr. J F Doré (Institut National de la Sante et de la Recherche Médicale-INSERM, Unit 218, Lyon, France). All the cell lines were grown in minimum essential medium with Earle's salts (Gibco-BRL, Paisley, Scotland) supplemented with 10% fetal calf serum (Sigma-Aldrich), 1× solution of vitamins (Gibco-BRL), 1 mM sodium pyruvate (Gibco-BRL), 1× non-essential amino acids (Gibco-BRL) and 2 mg of gentamicin base (Gibco-BRL). Cells were cultured in a humidified atmosphere at 37° C. in 5% CO₂.

### Evaluation of the Maximal Tolerated Dose In Vitro

The determination of the maximal tolerated dose (MTD) in vitro is defined as the higher concentration which do not induces cell growth inhibition. The cells were plated at a density of 5×10³ cells per well in 96-well microplates (Nunclon™, Nunc) in 100 μl of culture medium and were allowed to adhere for 16 h before treatment. 100 μl of culture medium containing β-caryophyllene were added and incubated at 37° C. for 48 h. All compounds were dissolved in ethanol and the final concentration of ethanol in the culture medium was maintained at 0.25% (v/v). The effect of β-caryophyllene on the proliferation of tumour cells was assessed using resazurin reduction test as described below.

### Resazurin Reduction Test

The resazurin reduction test (RRT) was carried out according to the protocol as described by O'Brien et al. Briefly, plates were rinsed by 200 μl PBS (37° C., Gibco) at 37° C. using an automatic microplate washer (Cell Wash™, Labsystems, Helsinki, Finland) and emptied by overturning on absorbent toweling. Then, 150 μl of a 25 μg/ml solution of resazurin in MEM without Phenol red was added in each well using an automatic microvolume dispenser (Multidrop 384™ Labsystems). The plates were incubated 1 h at 37° C. in a humidified atmosphere with 5% of CO₂ for fluorescence development by living cells. Fluorescence was then measured on the automated 96-well plate reader Fluoroskan Ascent FL™ (Labsystems) using an excitation wavelength of 530 nm and an emission one of 590 nm. The fluorescence is proportional to the number of living cells in the well.

### Evaluation of the Maximal Tolerated Dose In Vivo

The maximal tolerated dose in vivo is defined as the higher concentration which is not toxic to the patient (mice or human).

In order to determine the maximal tolerated dose in vivo, an increasing dose of β-caryophyllene has been administered to B6D2F1 6-weeks old males mice. A minimum of 6 mice were tested by group. The principals criterions for toxicity were: 1) weight loss; ii) hair bristling; iii) stooped back; iv) hollow-eyed; v) instable walking; vi) diarrhea; vii) convulsions or shaking; and viii) dead.

### Analysis of Membrane Transport Alteration Using Calcein-AM

Membrane transport alteration was evaluated using calcein fluorescent dye accumulation inside of the cells. Briefly, L-929 cells were plated at a density of 1×10⁴ cells per well in 96-well microplates (Nunclon™, Nunc) in 100 μl of culture medium and incubated overnight at 37° C. The cells were washed with PBS 1× and incubated for 1 h with 100 μl of MEM containing 16 μM of calcein-AM, without Phenol red, in the presence or the absence of β-caryophyllene. Fluorescence was measured on the automated 96-well plate reader Fluoroskan Ascent FL™ (Labsystems) using an excitation wavelength of 485 nm and an emission wavelength of 530 nm.

Evaluation of Synergistic Effect of Terpenes in Combination with Antitumor Agents

Effect of terpenes as synergist in combination with antitumor agents. Briefly, the cells were plated at a density of 5×10³ cells per well in 96-well microplates (Nunclon™, Nunc) in 100 μl of culture medium and were allowed to adhere for 16 h before treatment. Then, 100 μl of culture medium containing growing concentration of paclitaxel with or without 12.5 or 200 μM of β-caryophyllene were added and incubated at 37° C. for 48 h. The compounds were dissolved in ethanol or DMSO and the final concentration of ethanol or DMSO in the culture medium was maintained at 0.25% (v/v). The proliferation of tumour cells was assessed using resazurin reduction test as described above. The synergistic effect of β-caryophyllene was determined by the comparison between the percentage of cells growth inhibition induced by the paclitaxel used alone and the one induced by the combination of paclitaxel and β-caryophyllene.

### Results and Discussion

### In Vitro and In Vivo β-Caryophyllene Toxicity

The in vitro toxicity of β-caryophyllene has been evaluated from 5 cell lines: human adenocarcinoma, MCF-7; human prostatic adenocarcinoma, PC-3; human lung carcinoma, A-549 and human colon adenocarcinoma, DLD-1 and from human and mouse fibroblasts.

Cell survival has been evaluated after receiving an increasing dose of β-caryophyllene between 6.25 and 800 μM. It is shown in table 1 that β-caryophyllene is not toxic in vitro in a dose less than 800 μM.

The in vivo toxicity has been evaluated in B6D2F1 mice. Between 250 to 7500 mg/kg has been administered, intraperitonealy, to the mice. The results obtained demonstrate that the β-caryophyllene is not toxic in the mouse at dosage more than 500 mg/kg. Studies performed by the National Cancer Institute has already demonstrated that β-caryophyllene is not mutagenic.

### In Vitro Synergistic Effect Between β-Caryophyllene and Paclitaxel

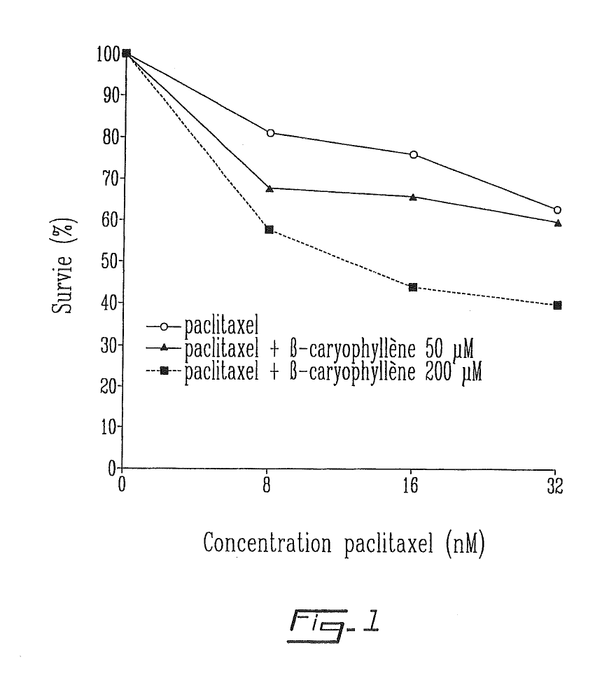

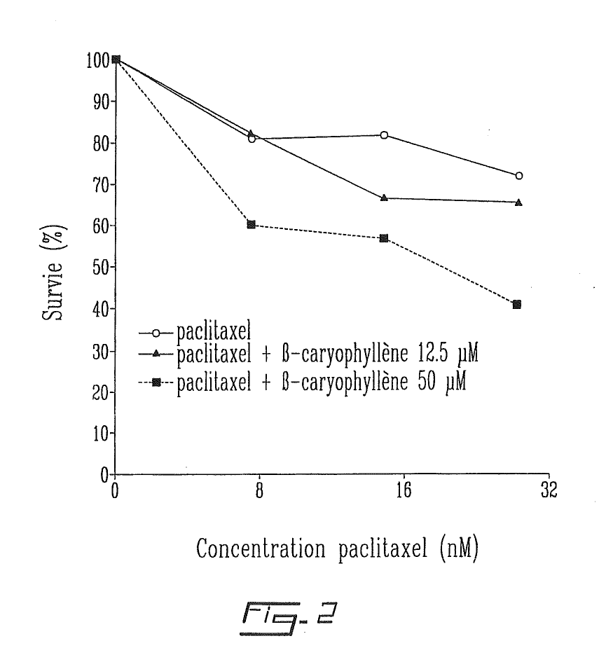

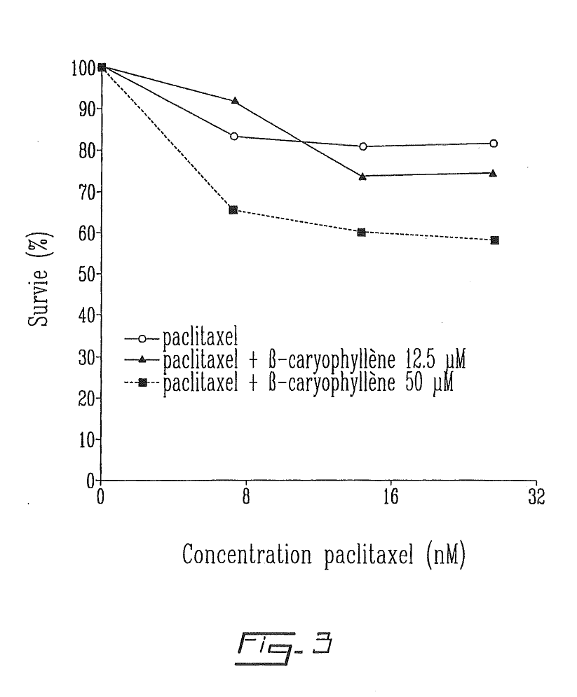

The results shown in FIG. 1 are indicating that the combination β-caryophyllene and paclitaxel is more efficient than the paclitaxel alone to kill MCF-7 cells. Similar results were obtained for colon adenocarcinoma cells (DLD-1) (FIG. 2) and mice fibroblasts (L-929) (FIG. 3).

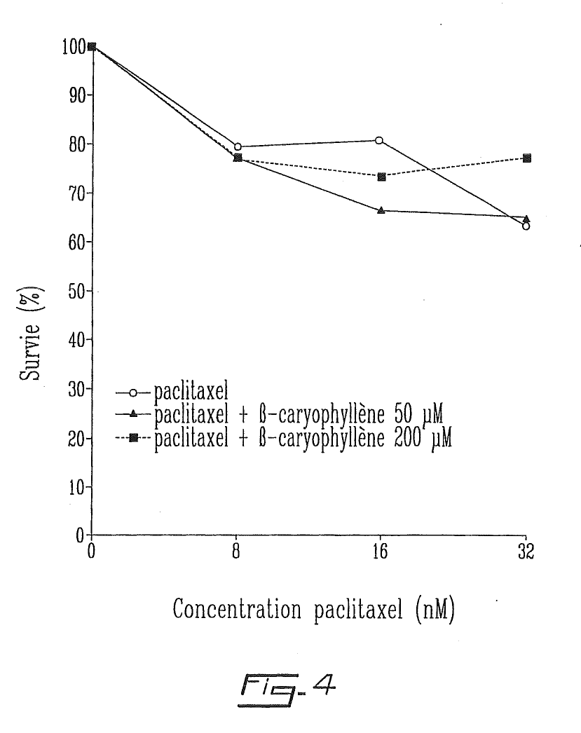

The results shown in FIG. 4 are indicating that the combination β-caryophyllene-paclitaxel is not having an enhanced effect in comparison with β-caryophyllene alone. This shows that the synergistic effect is directed to cancerous cells.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1.-16. (canceled)

17. A potentiator composition for enhancing therapeutical effect of an antitumoral agent, said composition comprising a sesquiterpene or derivative thereof in association with a pharmaceutically acceptable carrier, wherein said sesquiterpene is an amount sufficient to provide an effect in the presence of said antitumoral agent whereby said composition has no effect in absence of said antitumoral agent, with the provisio that the sesquiterpene or derivative thereof is not torilin.

18. The composition of claim 17, wherein said sesquiterpene is β-caryophyllene.

19. The composition of any one of claims 17-18, further comprising said antitumoral agent.

20. The composition of claim 19, wherein said antitumoral agent is from a class selected from the group consisting of alkylating agent, antimetabolite, antimitotic, antibiotic, immunotherapy and hormone.

21. The composition of claim 20, wherein said alkylating agent is selected from the group consisting of melphalan, cyclophosphamide, lomustine, carmustine and cisplatine.

22. The composition of claim 20, wherein said antimetabolite is selected from the group consisting of 5-fluorouracil, cytarabine and methotrexate.

23. The composition of claim 20, wherein said antimitotic is selected from the group consisting of paclitaxel, vincristine, vinblastine and vindesine.

24. The composition of claim 20, wherein said antibiotic is selected from the group consisting of doxorubicin, aclarubicin, daunorubicin and mitomycin C.

25. The composition of claim 20, wherein said immunotherapy is selected from the group consisting of vaccine, cytokine and interleukine.

26. The composition of claim 20, wherein said hormone is selected from the group consisting of steroid and glucocordicoid hormone.

27. The composition of claim 17, wherein said antitumoral agent is paclitaxel.

28. A method for enhancing the therapeutical effect of an antitumoral agent in a patient comprising the step of administering a therapeutically effective amount of the potentiator composition of any one of claims 17-18 in combination with said antitumoral agent to said patient.

29. The method of claim 28, wherein said administering of said composition is simultaneous, together or separately, consecutively and shortly prior to or after administering said antitumoral agent.

30. A method for enhancing the effect of an antitumoral agent in a patient comprising the step of administering a therapeutically effective amount of the composition of claim 19 to said patient.

31. The method of claim 28, wherein said administering is performed intravenously, orally, intraperitoneally, topically, subcutaneously, transdermally, intramuscularly, nasally, aerosolly, rectally and sublingually.
